(19) United States
(12) Patent Application Publication
(10) Pub. No.: US 2004/0253732 A1
(43) Pub. Date: Dec. 16, 2004

(54) Cloning vectors for homologous recombination and method using same

(76) Inventors: Christine Lapize-Gauthey, Vienne (FR); Alexandre Fraichard, Versailles (FR)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 10/479,497

(22) PCT Filed: May 28, 2002
(86) PCT No.: PCT/FR02/01782

(30) Foreign Application Priority Data
May 28, 2001 (FR) 0106921

(51) Int. Cl. C12N 15/74

(52) U.S. Cl. 435/471; 435/320.1

(57) ABSTRACT

The invention concerns novel vectors for use both in creating genomic DNA banks and as vectors for carrying out homologous recombination reactions in host cells, in particular for improving selection of said homologous recombination events, and decreasing the time for obtaining a final vector for performing the homologous recombination reaction. The invention also concerns a method using said vectors.

The present invention relates to novel vectors which can be used, at one and the same time, for creating genomic DNA libraries and as vectors for performing homologous recombination reactions in host cells, making it possible, in particular, to improve the selection of said homologous recombination events and to decrease the time for obtaining the final vector for effecting the homologous recombination reaction. The invention also relates to a method which employs these vectors. These novel vectors, and the method which uses them, are especially adapted for homologous recombination in mammalian stem cells.

The ability to obtain mice which carry genetic modifications which are programmed by the experimenter has completely altered the study of almost all aspects of the biology of this animal and of these different systems (immune system, nervous system, hematopoietic system, etc.). Furthermore, these methods for altering the genotype of the mouse make it possible to create murine models of human genetic diseases which are invaluable for studying the physiopathology of these diseases and possibly developing appropriate therapies. This has been made possible thanks to the fortunate conjunction of two very different approaches: the one approach has resulted in the in-vitro isolation of embryonic stem cells (ES cells) and the other has made it possible to identify the conditions which are required, in the cells of higher eukaryotes, for the process of homologous recombination between a known exogenous DNA and the homologous sequence in the chromosome.

Homologous Recombination in ES Cells: Creation of Null Mutations (Knockout Mice)

Studies carried out in the 1980s (Smithies and Capecchi, for example) demonstrated that embryonic stem (ES) cells possess the enzymic apparatus which is required for homologous recombination (HR) between an exogenous DNA sequence and the homologous sequence which is present in the genome of the mouse, even if this homologous recombination is a rare event as compared with the random integration of this same DNA fragment (Smithies et al., 1985; Wong and Capecchi, 1986). Despite this rare frequency of appearance, it has been possible to extend the technique of homologous recombination to the study of a large number of genes (Jackson laboratory) thanks to a variety of selection and screening stratagems.

Knockin: the Other Aspect of Homologous Recombination

An interesting variant of the targeting vectors for obtaining null mutations results from introducing a cDNA of interest in phase with the sequence encoding the target gene.

After the homologous recombination, the cDNA which has been inserted into the target gene is expressed under the control of the endogenous promoter. The choice of the cDNA depends on the project and on the sought-after goal. For example, the cDNA can be the sequence encoding a reporter gene such as β-galactosidase. The expression of this protein then mimics the expression of the targeted gene, thereby making it possible to determine its expression profile (Li et al., 1997) and/or to follow the destiny of the cells which are expressing the targeted gene (Schneider-Maunoury et al., 1993; Tajbakhsh et al., 1996).

From Null Mutations to Subtle Mutations

Even if null (knockout) mutations represent a very powerful instrument of genetic analysis, it is clear that other types of more subtle mutation (point mutations, small deletions or insertions) are very useful for refining study models. When mutated alleles are created, it is especially necessary to eliminate the selection sequences which could interfere with the regulation of the expression of the target gene or of adjacent genes (Fiering et al., 1999). Several strategies can be used for creating “clean” mutations (Moore et al., 1998; Cohen-Tannoudji and Babinet, 1998) such as the “plug and socket” strategy (Detloff et al., 1994), the “hit and run” strategy (Hasty et al., 1991) or the Cre/loxP or FLP/FRT recombinase system (Kilby et al., 1993).

The recombinase (Cre/loxP, FRT/FLP, etc.) strategy is based on using a protein, i.e. the recombinase, and target sequences. In the Cre/loxP system, the Cre protein is a recombinase which was identified in the bacteriophage P1 and which acts when it recognizes a 34 bp sequence termed loxP in a DNA segment (Sauer and Henderson, 1988). After Cre has excised the fragment located between the two loxP sequences, the mutated allele then carries a loxP site. No interference of this site with gene expression has been demonstrated.

Conditional Mutagenesis

The Cre/loxP strategy (or FRT/FLP strategy, which is similar) enables a mutation to appear conditionally in an animal during the course of development or in the adult (Cohen-Tannoudji and Babinet, 1998; Gu et al., 1994). In the first place, it is a matter of creating mice which carry an allele which is flanked by two loxP sequences which frame an essential part of a gene of interest without, for all that, altering its function by, for example, locating the loxP sequences in introns (“floxed” gene). A mouse which has been produced in this way is then coupled with another, transgenic mouse which is expressing the recombinase in a particular cell type. This strategy is potentially very effective since it makes it possible not only to circumvent the problem of the embryonic lethality which occurs when all the cells of the embryo carry the mutation but also to examine the effect of this mutation in any tissue provided that a line of transgenic mice expressing the Cre protein in the tissue in question is available (Xu et al., 1999; Shibata et al., 1997; Kulkarni et al., 1999, Tsien et al., 1996; Harada et al., 1999).

An additional refinement consists in controlling the induction of the mutation in time as well as in space using inducible systems. In this version, the Cre protein is expressed, together with a ligand-binding domain, in the form of a fusion protein. This fusion protein does not exhibit any Cre activity. On the other hand, in the presence of the appropriate ligand, a change in conformation restores the Cre activity. Thus, in transgenic mice which are carrying the two “floxed” alleles of the gene of interest and which are expressing the Cre fusion protein (ligand receptor combined with the protein) in a particular tissue (Shibata et al., 1997), administration of the appropriate ligand will bring about the induction of the null mutation in this type of tissue at the desired time. It is also possible to control the expression of the recombinase by using systems which make it possible to induce or repress the transcription of a reporter gene. The most well-documented system uses the properties of the operator/repressor pair of the bacterial tetracycline operon (tet) (Baron U. et al., 1999).

However, all these promising techniques suffer from two major drawbacks, i.e. the construction of complex vectors and the time which is absolutely necessary for producing and analyzing these murine models.

Thus, it is estimated that, nowadays, the time for obtaining a conditional knockin mouse or knockout mouse is from about 15 to 18 months. This is because, on average, of about 8 to 9 months are required for cloning, mapping and constructing the target vector, 1 to 2 months are required for culturing ES cells and, finally, 6 to 8 months are required for animal work and crossing.

The object of the present invention is to reduce the time required for molecular biological manipulations, which should, by using the vectors and/or methods according to the invention, be able to be conducted in 3 months instead of 8 or 9 months.

This saving in time is significant while exhibiting a lower risk of failure due to the elimination of the subcloning steps, and the decrease in the number of cloning steps, as a result of using the vectors according to the invention. It is estimated that using these vectors makes it possible to get below the 12 month mark for producing a murine model.

The purpose of the present invention is to provide a novel basic vector which is suitable for creating a genomic DNA library and with said vector also being suitable for being transformed directly (without subcloning) for the purpose of effecting homologous recombination reactions in the target host. The invention also discloses a method for accelerating the construction of the vectors, which are used for carrying out homologous recombination in any organism and preferably in eukaryotic cells, in particular ES stem cells derived from multicellular organisms.

Thus, in a first aspect, the present invention relates to a cloning vector which is suitable for creating a genomic DNA library, characterized in that it exhibits a cassette comprising:

a polylinker for integrating a genomic DNA fragment, said polylinker being flanked by rare restriction sites, and said cassette being flanked by very rare restriction sites.

An element is “flanked by restriction sites” when at least one restriction site is present at each end of the element.

In a preferred embodiment, said cassette also comprises at least one negative selection gene.

The vector according to the invention is perfectly suited for creating a genomic DNA library and makes it possible, in particular, to clone medium-sized, and indeed large-sized, DNA fragments. The skilled person knows what is meant by a vector being suitable for creating a DNA, in particular genomic DNA, library, i.e. one that allows DNA sequences to be inserted and ensures the stability of these inserted sequences, in particular as regards the maintenance of the vector in the host cell, in particular during replication, and for which the level of recombination events which the DNA fragment undergoes with itself, and/or of loss of exogenous DNA sequences, is the level seen to be low. Examples of vectors which can be used are given below.

“Fragments of medium size” is understood, in particular, as meaning fragments of a size greater than 15 kilobases (kb), preferably of between 15 kb and 75 kb, preferably of between about 17 kb and about 50 kb, preferably of between about 20 kb and about 30-35 kb.

“Rare restriction sites” is understood as meaning restriction sites whose cutting frequency is greater than 10 kb, preferably 15 kb, more preferably 20 kb, in the human genome. Rare enzymes which may be mentioned, in particular, are PmeI, SgrAI, RsrII, ClaI, NotI, PacI, SrfI, NheI, FseI, NsiI.

A restriction site is termed “very rare” when its cutting frequency in the human genome is greater than 100, preferably 200 kb (for example AscI). Very rare restriction sites which can be used and which may be mentioned are the “homing endonucleases”. These enzymes are proteins which are encoded by genes possessing self-splicing introns. These enzymes make site-specific cuts in the double-stranded DNA and generally recognize sites of 18-20 bases or more. The following are noted, in particular: I-PpoI, I-CeuI, PI-PsI, I-SceI, PI-SceI.

Furthermore, as will be seen, the cloning vector according to the present invention is, particularly when it exhibits at least one negative selection gene, also particularly well suited for homologous recombination in mammalian cells, in particular murine, rabbit, porcine, bovine or human stem cells.

In one particular case, the vector according to the invention possesses a maximum of 4, preferably 3, preferably two, restriction sites in its polylinker for cloning the exogenous DNA. It is particularly advantageous to reduce the number of restriction sites which are located in the polylinker employed for inserting the exogenous DNA fragment, or which are located in the skeleton of the vector, in order to be able to use the corresponding restriction enzymes, after the exogenous DNA fragment has been cloned, for verifying, in particular, the nature of said fragment, if not to say its orientation, as well as the nature, orientation and precise location of the modifications which have been introduced into the exogenous DNA fragment. Thus, it is possible for the polylinker only to possess one single restriction site.

Nowadays, different types of vector exist for constructing DNA libraries. Mention may be made, in particular, of cosmids or artificial chromosomes. These latter, which can be adapted to bacteria (BACs), to yeasts (YACs) or to mammalian cells (MACs) permit the stable integration of DNA fragments which can be up to 200 kb or more in size. While cosmids have a more limited capacity (about 20 to 40 kb), this capacity is nevertheless greater than that of the classic plasmids, for example those derived from pBlueScript, which can only accommodate about 14-15 kb at most.

Bacterial artificial chromosomes are particularly attractive vectors since they can be used in hosts (in particular Escherichia coli) which are easy to manipulate and ensure that the DNA fragments which have been introduced are maintained stably.

Thus, in one particular embodiment, the vector is derived from a cosmid or from an artificial chromosome vector, preferably a bacterial artificial chromosome vector.

A “vector derived from another vector” is understood as meaning that, taken overall, the final vector possesses the same skeleton (in particular the origins of replication, etc.) as the starting vector from which it is derived (parent vector). It is important to note that this also preferably means that the intergenic elements are conserved. The changes which are made in the basic vector are such that the replication host for the vector remains the same and the changes do not alter the fundamental properties of the vector (copy number, insert size, stability, etc.). It is to be noted that it is possible to envisage altering the selection gene of the derived vector as compared with that of the parent vector as long as the exogenous DNA-receiving properties of the derived vector are not altered as compared with the parent vector.

Preference is given to selecting a vector which is derived from the body of the bacterial artificial chromosome vector pBe1oBAC11, which is well known to the skilled person and whose sequence can, in particular, be obtained in GenBank under the accession number U51113.

The vector according to the invention is preferably based on the pBe1oBAC11 skeleton which has been digested with SalI and religated. Thus, the vector according to the invention has lost the sequences cosN, loxP and lacZ which were initially present in pBe1oBAC11. The reason for this is that such a modification deletes certain sequences which are of bacterial origin (lacZ) or are of use in yeast (cosN) or may subsequently interfere with a recombinase which is of use for the method according to the invention (loxP). Thus, the deletion of the components of bacterial or yeast origin is advantageous for improving the frequency of the recombination events in mammalian cells. Preserving the loxP site could turn out to be a problem as regards subsequently using a Cre recombinase on the resulting vectors.

In one particular embodiment, at least one restriction site, selected from the sites which are present 1, 2 or 3 times in the skeleton of the parent vector, has been deleted in the vector according to the invention.

The absence of at least one of the abovementioned restriction sites in the skeleton of the vector according to the invention makes it possible, in particular, to use this site for studying the fragments which are integrated into said vector or for using an enzymic method to introduce the selection genes which alter the exogenous DNA fragment before it is used for the actual homologous recombination.

The modification of the skeleton of the vector, for the purpose of deleting the abovementioned sites, can be effected by site-directed mutagenesis, so as to ensure that the corresponding restriction enzyme no longer cuts the skeleton of the vector.

In a preferred manner, and in particular when the vector according to the invention is derived from the vector pBe1oBAC11, at least one restriction site selected from ApaI, BstEII, SacII, SfiI, SpeI, SphI, StuI, XhoI, BssHII, EcORI, EcORV, KpnI, NdeI, NotI, NruI, PvuI, SgrAI, XbaI, PstI, SalI and SmaI has been deleted from its skeleton. Thus, as has been made clear above, it can be advantageous to delete as many of these restriction sites as possible, that is to say at least 2 of these sites, even better 3, 4 or 5, and, in the best case, not to retain any of them. In one specific case, the skeleton of the vector according to the invention no longer possesses the BstEII site.

In one particular embodiment, the cassette of the vector according to the invention possesses a very rare restriction site at one of its ends and two very rare restriction sites at the other end, with one of these two sites preferably being identical with the site which is located at the first end of the cassette.

The presence of these sites makes it possible either to linearize the vector according to the invention (using the site which is present at only one end of the cassette) or to excise the insert which is integrated into the vector (using the site which is located at the two ends of the cassette).

In one particular and preferred case, the polylinker of the vector according to the invention, which enables the exogenous DNA to be integrated, is flanked by several (in particular a number equal to or greater than 2, 3 or 4) rare restriction sites which are selected, in particular, from PmeI, SgrAI, RsrII, ClaI, NotI, PacI, SrfI, NheI, FseI.

The vector according to the invention thus contains at least one negative selection gene. It is possible, in particular, to choose suicide genes, that is to say genes which encode products which are capable of transforming an inactive substance into a cytotoxic substance. Mention may be made, in particular, of the thymidine kinase genes of the HSV-1 virus, which can be used with ganciclovir or acyclovir, the DTA gene, which encodes the diphtheria toxin A fragment, which is described in Yagi et al., (1990, PNAS, 87, 9918-22) and which is toxic by its prescence alone. There are nowadays several suicide genes which are available to the skilled person and which can be used as negative selection markers. Rat cytochrome p450 and cyclophosphamide (Wei et al., 1994, Human Gene Ther. 5, 969-978), Escherichia coli (E. coli) purine nucleoside phosphorylase and 6-methylpurine deoxyribonucleoside (Sorscher et al., 1994, Gene Therapy 1, 223-8), E. coli guanine phosphoribosyl transferase and 6-thioxanthine (Mzoz et al., 1993, Human Gene Ther. 4, 589-595) and cytosine deaminase (CDase) or uracil phosphoribosyl transferase (UPRTase), which latter two can be used with 5-fluorocyto-sine (5-FC), may also be mentioned.

Thus, while the negative selection gene is located at one end of the polylinker which is used for cloning the genomic DNA fragment, the vector according to the invention preferably contains two negative selection genes (which may possibly be identical), with these genes being located on either side of said polylinker.

In a preferred manner, the cloning vector according to the invention contains two copies of the same negative selection gene, with these copies flanking the polylinker which is used for cloning the genomic DNA fragment and which is itself flanked by rare restriction sites (FIG. 1).

This therefore results in a construct which is made up as follows (from 5′ to 3′):

at least one very rare restriction site,

a negative selection marker,

an SR1 cassette possessing at least one (preferably several) rare restriction site(s),

the polylinker exhibiting a reduced number of restriction sites,

an SR2 cassette containing at least one (preferably several) rare restriction site(s), with these sites preferably being different from the site(s) of the first cassette,

a negative selection marker, which may possibly be identical to the first marker,

at least one very rare restriction site, preferably two very rare restriction sites, with one preferably being identical to the first site mentioned above, and with the other being different.

Thus, the presence of several very rare restriction sites makes it possible to linearize the cloning vector according to the invention or to isolate the insert located between two very rare restriction sites.

It is to be noted that preference is given to the rare restriction sites which are present in one of the cassettes all being different from those present in the other cassette. Thus, this makes it possible to linearize the vector according to the invention after cloning the genomic DNA fragment and, in as much as it is of interest for homologous recombination events in mammalian cells to have available a long homology arm and a short homology arm, to be able to use exonuclease III in order to reduce one of the two arms. It is to be noted that the cassettes SR1 and SR2 can also contain less rare restriction sites such as, in particular, SacI, SwaI, SphI or SalI.

The vector according to the invention therefore makes it possible to construct a genomic DNA library from the organism in which it is desired to carry out the homologous recombination. This library is preferably composed of fragments of medium size so as to be able to carry out the modifications which are required for the homologous recombination directly in the vector which has been used for constructing the library without it being necessary to subclone the genomic DNA fragment carrying the target locus after it has been identified in the library. It is nevertheless important to note that the size of the fragments in the library can be proportional to the size of the genome of the target organism in as much as it is of interest (in particular for practical reasons of manipulation) to limit the number of clones which are present in the library. Thus, in the case of a genome such as that of the mouse, the library of choice is one which is constructed from fragments having a mean size equal to 20-30 kb.

In one preferred embodiment, the vector according to the invention is the vector depicted by SEQ ID No. 1, additionally possessing the following characteristics: 13478 bp, circular, nt 4629.5597 SOPB protein, nt 3454 . . . 4626 SOPA protein, nt 2120 . . . 2872 REP protein, nt 999 . . . 1343 resolvase protein, nt 114 . . . 780 gene for resistance to chloramphenicol, nt 6443 . . . 9851 DTA gene, nt 10037 . . . 13451 DTA gene, nt 9943 . . . 9962 Sp6 oligonucleotide, nt 9902.9922 T7 oligonucleotide.

The present invention therefore also relates to a genomic DNA library of fragments of medium size, which library is constructed in a vector according to the invention. The skilled person is familiar with the different means for constructing a genomic DNA library in an appropriate vector, in particular by partially digesting the genomic DNA.

The vector according to the invention has furthermore been optimized in order to be perfectly suited to a method for implementing targeted recombination in the genome of a target organism using a library of genomic fragments which are cloned therein.

Thus, it is possible to introduce the elements which modify the fragment which carries the target locus, and which has been identified in the genomic DNA library, directly in the vector which carries the fragment and to perform the homologous recombination reaction in the target host without having any need to subclone the target locus.

This is because the fact that the vector according to the invention carries a negative selection gene makes it possible to more easily select the homologous recombination events.

After the clone carrying the target locus has been selected in the genomic DNA library, it is possible to introduce the DNA fragment(s) which will be used for modifying the target gene in vivo (for example the positive selection genes or Cre recombinase recognition sites). These DNA fragments can be introduced into the vector by means of standard cloning (use of enzyme sites) or by means of homologous recombination in bacteria or else by means of using transposons or by means of any other suitable technique. The use of transposons is preferred because it makes it possible to reduce the time which is required for obtaining the modified locus since there is no need to study the cartography of said locus beforehand.

Thus, the present invention also relates to a method for performing a targeted recombination in an organism, starting with a genomic DNA library in a vector according to the invention, which method comprises:

a) incubating a vector of said genomic library with at least one DNA fragment which comprises at least one transposon, with said transposon comprising

a positive selection gene,

where appropriate, a negative selection gene and/or a marker gene,

with said selection genes being flanked, where appropriate, by the sites of action I of a site-specific recombinase I and, optionally,

at least one site of action II of a site-specific recombinase II which is different from the first recombinase I,

with said site of action II not being located between said sites of action I of the first recombinase I,

in the presence of an enzyme possessing a transposase activity for said transposon(s), such that said transposon(s) is/are transferred into said genomic fragment,

b) introducing said vector, which is, where appropriate, linearized and which is carrying said genomic fragment into which said transposon(s) is/are inserted, into a target host cell derived from said organism,

c) selecting homologous recombination events in said target cell by using the positive selection gene(s) carried by said transposon(s) and, where appropriate, the negative selection gene carried by said cloning vector.

Preferably, said modified genomic fragment is a fragment of medium size.

The method according to the invention permits targeted integration and homologous recombination in a target organism, preferably a mammalian organism, more preferably rodent (in particular murine) cells, rabbit cells, porcine cells, ovine cells, bovine cells and even human cells.

In order to ascertain the integration loci of the transposons in the genomic fragments carried by the vectors according to the invention, it is possible, in particular, to use the restriction enzymes which cut at the rare sites in the cassettes SR1 and SR2. These enzymes are also used, where appropriate, for linearizing the vector and to form the short and long homology arms due to the targeted action of exonuclease III.

The method according to the invention is advantageously completed by following step c) with a step d):

d) subjecting the cells which have been transformed by homologous recombination and selected in step c) to the action of the recombinase I in order to eliminate the positive and, where appropriate, negative selection gene(s) carried by the transposon(s).

Preferably, the target organism is therefore a multicellular organism and said target cell derived from said organism is a stem cell.

In a preferred case, the deletion of a genomic fragment and/or the insertion of an exogenous fragment in said organism is induced after step c) or, where appropriate, step d) by bringing the cells selected in step c), or derivatives of said cells, into contact with the recombinase II. This thus makes it possible to obtain conditional inactivation of the gene which has been targeted, in particular when the construct has been produced such that the loxP sites come to be located in introns of the gene which it is desired to inactivate. This conditional inactivation is effected in vivo as described in the introduction.

The present invention also relates to a kit which comprises:

a cloning vector according to the invention,

at least one DNA fragment which comprises at least one transposon, with said transposon comprising a positive selection gene and, where appropriate, a negative selection gene and/or a marker gene, with said selection gene(s) being, where appropriate and preferably, flanked by the sites of action I of a site-specific recombinase I.

Optionally, said transposon also comprises a negative selection gene or a marker gene which is located adjacent to the positive selection gene, which may or 35 may not be located between the sites of action I of said site-specific recombinase I when this latter is present.

The transposon which can be used for the present invention can be of any kind, in particular the transposon Tn5. The transposon Tn5 is preferably selected because it integrates unidirectionally into the target DNA (from 5′ to 3′), because it can be used directly with a transposase without it being necessary to add cofactors, and because it is marketed in a kit.

In order to circumvent the problem of instability, if it arises, it is possible to envisage using different types of transposons (for example Tn5, Tn10 or Tn7) (Brune et al., 1999; Chatterjee and Coren, 1997; Goryshin et al., 2000; Goryshin and Reznikoff, 1998; Stellwagen and Craig, 1997; Westphal and Leder, 1997; Yang et al., 1997).

Some negative selection genes which can be used for implementing the present invention have been mentioned above. Marker genes which can be used and which may be mentioned are the lacZ gene or the genes encoding fluorescent proteins (FPs).

The positive selection genes are well known to the skilled person and are preferably genes for resistance to an antibiotic, such as the genes for resistance to kanamycin, which also provides resistance to neomycin in mammalian cells, for resistance to hygromycin, for resistance to zeocin, for resistance to blasticidin, etc.

Optionally, the transposon also comprises at least one site of action II for a site-specific recombinase II which is different from the first recombinase I, with said site(s) of action II not being located between said sites of action I of the first recombinase I.

Preferably, the kit according to the invention comprises two DNA fragments, with each one comprising a transposon and with each transposon carrying a different positive selection gene and, where appropriate, a negative selection gene, with the negative selection gene then preferably being identical in the case of the two transposons, with said positive and negative selection genes preferably being flanked by the sites of action of a recombinase I.

In a preferred embodiment of the invention, each transposon comprises a sequence which corresponds to a site of action of a recombinase II which is different from the first recombinase I.

Thus, in a preferred case, said recombinase I is the recombinase FLP, with said recombinase II being the recombinase Cre. It is then advantageous for one of the transposons to contain the native FRT sequences which are recognized by the recombinase FLP, with the other transposon then containing the mutated FRT* sequences as described by Schalke and Bode (1994).

In order to implement the method according to the invention, as described above, for facilitating the targeted recombination events, it is advantageous for the kit according to the invention to also comprise an enzyme which possesses a transposase activity for said transposon(s) as well as the implementation instructions for inserting said transposon(s) into the genomic fragment which is located in said cloning vector.

### DESCRIPTION OF THE FIGURES

FIG. 1: Diagrammatic map of a modified vector according to the invention. SR1 and SR2: cassettes exhibiting rare restriction sites. PL: polylinker for cloning the genomic fragments. DTA: gene for the diphtheria toxin A subunit.

FIG. 2: Description of the insertion of two transposons into a DNA fragment in the vector according to the invention. DTA: gene for the diphtheria toxin A fragment which is present in the vector according to the invention. SR1 and SR2, cassettes containing at least one rare restriction site. Very rare site: in particular, homing endonuclease site. Kana: gene for resistance to kanamycin in bacteria and for resistance to neomycin in eukaryotic cells. Zeocin: gene for resistance to zeocin. TK: gene for the HSV1 thymidine kinase. FRT: recognition sites for the recombinase FLP. FRT*: mutated recognition sites for the recombinase FLP. LoxP: recognition sites for the recombinase Cre. OE/S: sequences enabling the integration locus of the transposon to be determined.

FIG. 3: Diagram of the homologous recombination of a vector according to the invention following integration of transposons into a genomic locus. The black boxes 1, 2, 3 and 4 represent the exons of the target gene. The other acronyms have the previous meaning. The homologous recombination event is selected by the resistance to neomycin (presence of the kanamycin gene) and to zeocin, and the absence of the DTA genes of the vector (negative selection).

FIG. 4: Action of the FLP recombinase in the cells selected for the homologous recombination in order to obtain a final conditional knockout locus by deleting the exogenous DNA sequences (resistance genes).

FIG. 5: Action of the Cre recombinase on the cells selected for the homologous recombination in order to obtain a final knockout locus and production of a truncated protein.

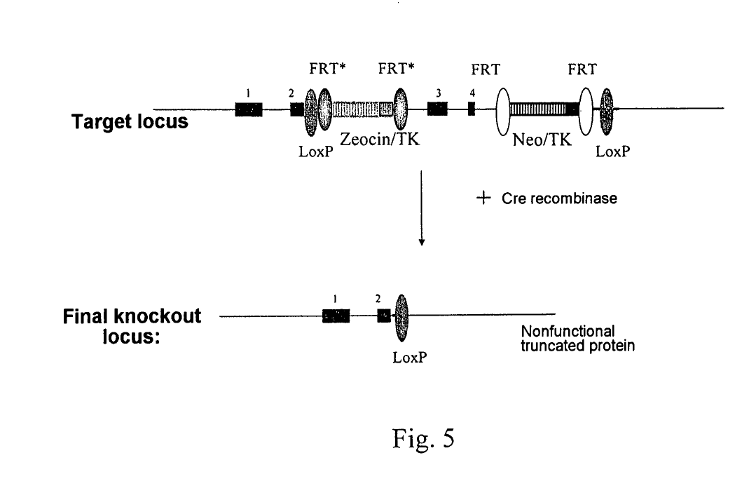

FIG. 6: Action of the Cre recombinase in vivo in the transgenic animal derived from the cells which were subjected to the FLP recombinase in order to obtain a final knockout locus and conditional production of a truncated protein.

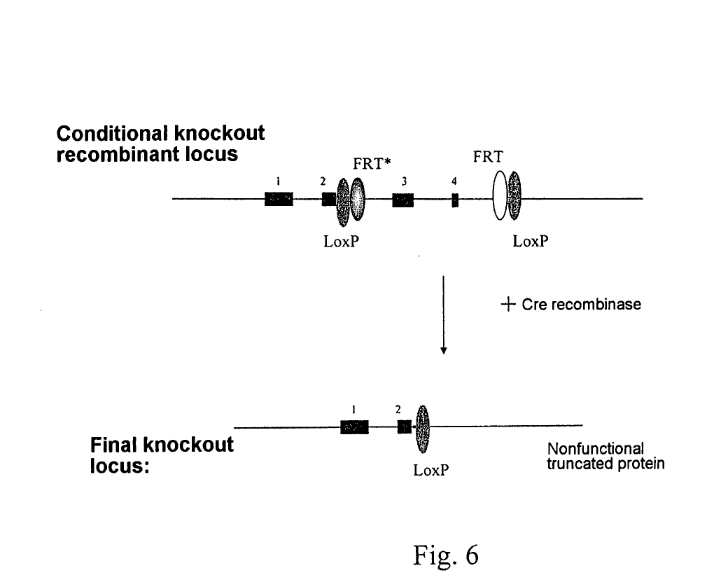

FIG. 7: Map of the plasmid pBe1oBAC11, which is used as parent vector for some vectors according to the invention.

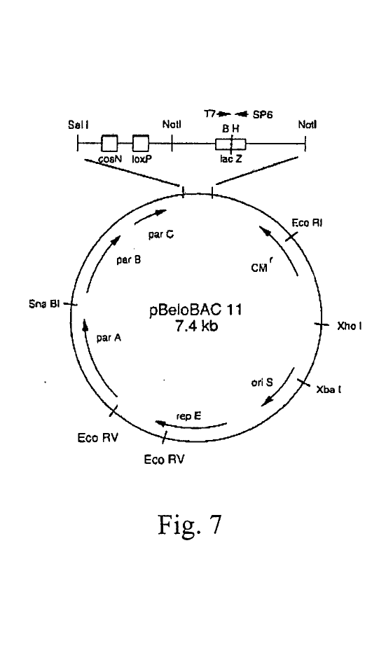

FIG. 8: Strategy for pooling the clones of the genomic DNA library which have been integrated into a vector according to the invention. 8.A: Pooling clones from 24 plates (superpooling). 8.B: Defining pools of lines, columns and individual plates. 8.C: PCR plate from a superpool. 8.D: plate for PCR from pools of lines, columns and plates.

### EXAMPLES

### Example 1

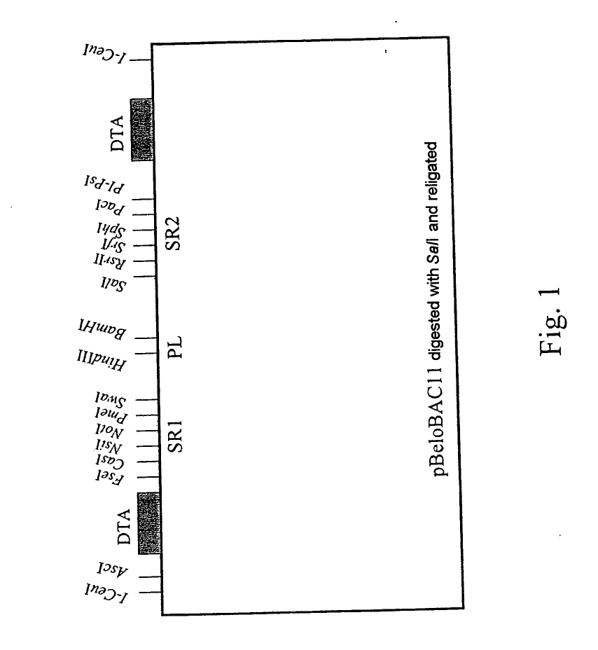

The first step consists in creating a mini-BAC (between 20 and 30 kb) library which is suited to constructing all types of homologous recombination vectors. This library is produced using mouse 129 sv genomic DNA and a modified pBe1oBac11 vector (FIG. 1). Negative selection genes are added on either side of the pBe1oBAC polylinker. The genomic FNA fragments known as the long homology arm and short homology arm of the homologous recombination vector (Thomas and Capecchi, 1987; Hasty et al., 1991; Thomas et al., 1992) are derived from this library.

Standard selection techniques such as PCR and Southern blotting are used for selecting the mini-BAC which contains the part of the gene under study which is to be modified.

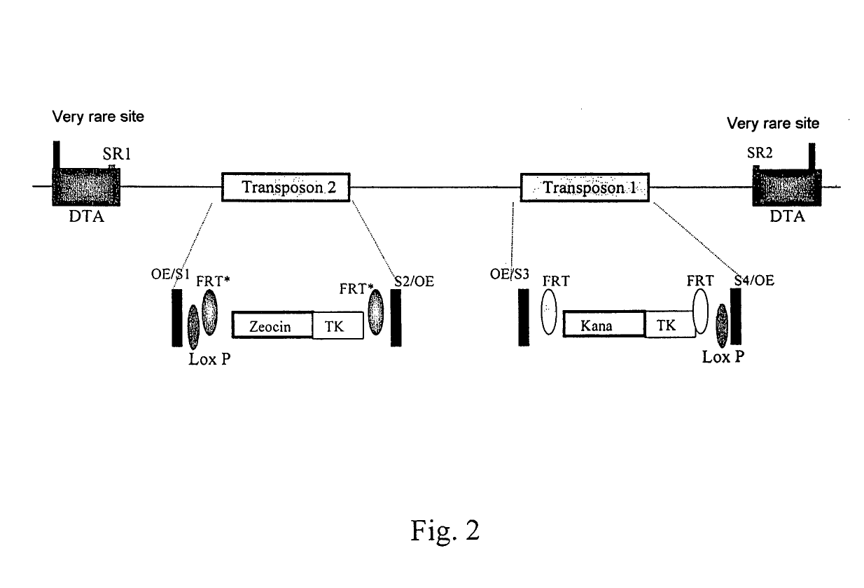

Transposons are inserted into the mini-BAC in order to introduce prokaryotic or eukaryotic selection genes and sites of action for the recombinases. These transposons also carry specific sequences which can be used for sequencing, for PCR tests and for enzymic digestions (FIG. 2).

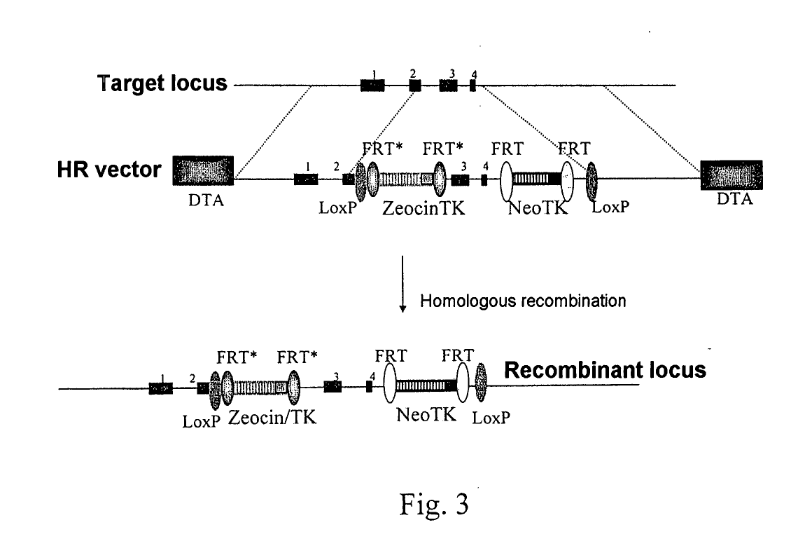

The homologous recombination vector is electroporated into ES cells and the homologous recombination event is selected in the presence of zeocin or G418 (Yagi et al., 1990) (FIG. 3).

What is termed the clean mutation is obtained in these recombinant ES cells by the action of the FLP recombinase in the presence of gancyclovir (Hasty and Bradley, 1993) (has the effect of eliminating the ES cells which contain the thymidine kinase gene, TK).

Constructing the BAC Library

The mouse genome is formed from approximately 3 thousand million base pairs. In order to cover this genome satisfactorily, it is essential to have a good representation of its diversity within the starting library. A statistical model was worked out with this aim in view. It is based on the following mathematical equation:

N=−L0 ln((1−p)/(L−1))

This equation takes into account the following elements:

L: size of the fragment which is inserted into the mini-BAC

N: number of clones making up the library

p: percentage of success in screening the library

1: size of the target locus before being present in the mini-BAC

L: minimum indivisible length of interest

L0: length of the genome.

The size of the genomic fragment is selected to be between 20 and 30 kb (mean at 25 kb). This size enables the BAC to be manipulated more easily while at the same time not increasing to too great an extent the number of clones which are required to make up the BAC library.

The first step consists in selecting the mini-BAC, or the mini-BACs, which are positive for the region of the gene of interest (target locus). This work is carried out by analyzing the entire library by combining simple PCR and Southern blotting selection techniques. On average, 150 PCRs are required for carrying out this work (testing of the superpools and then of the pools and finally identification of the positive clones).

Of the positive BACs which are detected, the one to be selected is that which contains the gene of interest in a suitable position. This is because it is essential that the insertion sites which are intended for integrating the transposons should not be too close to the ends. The short homology arm should have a minimum size of approximately 3.5 kb in order to allow the construction of the positive control vector and of the final homologous recombination vector. This choice is made by means of a simple enzymic profile followed by detection of the target fragments.

Integration of the Transposons

In the mini-BAC which is selected, two transposons (Tn5) are integrated randomly, with each carrying different resistance genes (Yang et al., 1997). Each transposon possesses specific sequences enabling it to fulfill its mission of insertion (OEs amounting to two times 19 bp). The insertions, and the characterization of the site of insertion of each transposon, are carried out consecutively.

Transposon 1 corresponds to the standard exogenous cassette of a homologous recombination vector (FIG. 2) containing the positive selection gene (Neo) and the negative selection gene (TK) which may or may not be flanked by sites of action of a recombinase, by marker genes (lacz, for example), etc. It is to be noted that this positive selection gene is also used as the gene for selecting the integration of the transposon into the mini-BAC (mixed eukaryotic/prokaryotic promoter).

For the usual inactivation of a gene, transposon 1 is introduced into a region which is essential for expression of the gene (exon 1, for example). If not, it will be introduced into an intron in order to enable subsequent conditional inactivation to be effected.

Transposon 2 supplies a second negative selection gene (TK) and a positive selection gene for resistance to zeocin, flanked by sites of action of a recombinase. This transposon makes it possible to introduce selection pressure on the integration of a loxP site (added or not from another sequence) used by the Cre recombinase. A large number of problems occurring during the homologous recombination event have been mentioned in relation to animal models of conditional gene inactivation. Thus, it is not unusual to have homologous integration of the part which is under the G418 (neomycin) selection pressure but on each occasion to lose the loxP sequence (34 bp) which is present upstream or downstream. The introduction of the second selection gene (zeocin) at the level of this loxP sequence increases the chances of obtaining ES cells which have homologously integrated the two transposons and therefore the two loxP sequences of interest.

The transposons are designed so as to be able to determine their relative position as well as their position in relation to the ends of the insert in the mini-BAC (restriction site (S1, S2, S3, S4), specific sequences for PCR primers (OE), etc.).

Selecting the Homologous Recombination Event

At this stage, a large number of antibiotics are used in combination. The integration of the two loxP sequences is effected under G418 and zeocin selection pressure. The selection of the homologous recombination event is facilitated by the negative DTA selection at the two ends of the construct.

“Cleaning” the Recombinant ES Cells: Obtaining a So-Called “Clean” Mutation

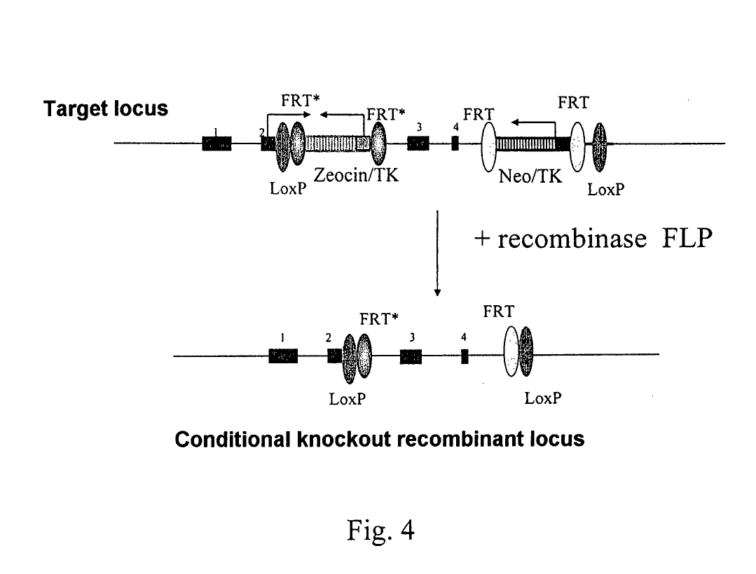

In order to produce a murine model which is as close as possible to reality, it is necessary to remove the genes which were used for selecting the events of the integration of the transposons into the mini-BAC and the event of the integration of the transgene into the ES cells (FIG. 4). In this regard, it is attractive to use sequences which are recognized by the FLP recombinase, i.e. the mutated or unmutated FRT sites.

The principle is as follows: two FRT sites will enable the FLP to excise the sequences which are located between these two sites. Only one of the two FRT sites then remains. A mutated FRT site can only act together with another mutated FRT site (Schlake and Bode, 1994).

In this way, it is possible to excise the genes used for selecting the insertion of the two transposons. The only sequences which then remain are those permitting the insertion of the transposons (OE), the two loxP sequences and the two FRT sequences (mutated or not) These sequences (OE, loxP and FRT) are located at two insertion sites, with each representing 150 bp (see FIG. 4). The so-called clean recombinant ES cells are microinjected into blastocysts in order to give rise to a murine model.

### Example 2

### Creating the Cloning Vector rTgV

Constructing the Vector rTgV

1) Construction of a derivative of pBe1oBAC11 which has lost the fragment contained between the sites SalI 7030 and SalI 646 as well as these SalI sites and which contains, in place of the fragment between the SalI 7030 and SalI 646 sites, the following intermediate cloning sequence (SEQ ID No. 2):

5′ctcgagtaactataacggtcctaaggtagcgaggcgcgccatcgatgtcgact cgctaccttaggaccgttatagttactcgag-3′

containing the following restriction sites: XhoI-ICeuI-AscI-ClaI-SalI-ICeuI-XhoI. The XhoI ends were ligated to the SalI ends of the vector.

The resulting plasmid is designated pBe1oBAC13.

2) Construction of a derivative of pBe1oBAC13 which has lost the BstEII site. The pBe1oBAC13 vector was digested with BstEII, treated with Klenow and religated.

The resulting plasmid is designated pBe1oBAC13 BK-1.

3) Construction of a derivative of pBe1oBAC13 BK-1 which contains a SwaI site in the intermediate cloning sequence. The vector pBe1oBAC13 BK-1 was digested with AscI and SalI and-the following sequence was inserted between these two sites:

5′-ggcgcgccatttaaatctcgag-3′

This sequence contains the following restriction sites: AscI-SwaI-XhoI. The XhoI end was ligated to the SalI end of the vector and the AscI end was ligated to the AscI end of the vector.

The resulting plasmid is designated pBe1oBAC13 BK-1 AS-1.

4) Construction of a derivative of the vector DTArTgV which contains a SmaI site in place of the SalI site at position 2 and a SalI site in place of the BamHI site at position 3049. This construction was performed by inserting double-stranded oligonucleotides containing the SmaI site at position 2 and the SalI site at position 3409 of the DTArTgV plasmid.

The resulting plasmid is designated DTA ASalI ABamHI.

5) Cloning two copies of the 3.4 kb fragment containing the DTA gene at the SwaI site of the vector pBe1oBAC13 BK-1 AS-1.

One copy of the DTA gene is derived from the vector DTArTgV as a result of digesting with BamHI, treating with Klenow, digesting with SalI and purifying the 3.4 kb fragment of interest. The other copy of the DTA gene is derived from the vector ΔTA ΔSalI ΔBamHI as a result of digesting with SmaI and SalI and purifying the 3.4 kb fragment of interest. Following this cloning, one of the SwaI ends of the plasmid pBe1oBAC13 BK-1 AS-1 is ligated to the BamHI end (Klenow) of a copy of the DTA gene and the other SwaI end is ligated to the SmaI end of the second copy of the DTA gene. The two copies of the DTA gene are linked by their SalI end.

The resulting plasmid is designated pBe1oBAC13 BK-1 AS-1 DTA-2.

6) Preparation of a plasmid which is derived from Bluescript KS(+) and which contains the following sequence:

5′-tcgagggccggccgagctcatgcattgcggccgcgtttaaacatttaaatgt aatacgactcactatagggcgaggatccaagcttagtattctatagtgtcaccta aatcgtatgtcgaccggaccggcccgggcgcatgcttaattaatggcaaacagct attatgggtattatgggtctcgag-3′

This sequence contains the following restriction sites in this order: XhoI-FseI-SacI-NsiI-NotI-PmeI-SwaI -BamHI-HindIII-SalI-RsrII-SrfI-SphI-PacI-PIPspI-XhoI as well as a so-called T7 sequence between the SwaI and BamHI restriction sites and a so-called SP6 sequence between the HindIII and SalI restriction sites. This sequence is designated [polylinker] in the subsequent description.

The resulting plasmid is designated BlueLC-2.

7) Cloning the “polylinker”, prepared by digesting the plasmid BlueLC-2 with XhoI and purifying the 187 bp fragment of interest, at the SalI site of the plasmid pBe1oBAC13 BK-1 AS-1 DTA-2.

The resulting plasmid is designated pBe1oBAC13 BK-1 AS-1 DTA-2 LC-16.

### Example 3

### Creating the BAC Library Using the rTgV Cloning Vector Described in Exmaple 2

This library consists of 300000 clones representing 2.5 times the murine genome. It was constructed using the following procedures:

Bacterial Strain and Culture Conditions

E. coli DH10B (Grant et al. 1990) was used as the host for the mini-BACs. The basic vector (genoway vector described above) was used for constructing this library. The DH10B bacteria are cultured in the usual manner in LB at 37° C. (Sambrook et al. 1989). The recombinant clones were selected on agar plates containing 12.5 μg of chloramphenicol/ml, using unfrozen electrocompetent E. coli DH10B bacteria as the starting material (Sheng et al. 1995).

Preparing Genomic DNA and Constructing the Library

The genomic DNA was prepared from a culture of mouse embryonic stem (ES) cells having a 129 svJ genetic background using the standard protocol described in Sambrook et al. (chapter 9) and in Vaiman et al. (1999).

The genomic DNA is partially digested with HindIII and the 20-30 kb fragments are isolated by pulsed field and introduced into the vector rTgV by means of HindIII ligation.

The rTgV vector was prepared using the Woo et al., 1994 protocol.

### Example 4

### Working Out the Structure of the BAC Library and its Mode of Screening

After the different clones making up the BAC library, containing the genomic DNA, have been obtained, the library is graded so that it can be rapidly screened for identifying the clone(s) carrying the locus of interest on which it is desired to perform the recombination operation.

The colonies are therefore picked and various steps of pooling the colonies are performed, thereby making it possible to carry out fewer screening reactions but even so identify the clones unequivocally.

Picking the Colonies

The colonies, which were previously spread on a 22×22 cm² tray, are picked using a Genetix ‘Qpix’ automated station and then deposited in 96-well plates (TPP brand, distributed by ATGC, ref. T92697) containing 200 μl of 2YT (Sigma, ref. Y2377)—10% (final) glycerol—12.5 μg of chloramphenicol/ml (final) (Sigma, ref. C0378) medium per well (MBAA plates 0001 to 3168 ‘A’). These plates are incubated overnight at 37° C.

Copies of the Library Plates

Two copies of these plates are made:

one copy in a 96-well plate which is identical to the parent plate

one copy in a 96-well plate which is of the “deep-well” type (ATGC ref. 219009) and which contains 1 ml of 2YT-chloramhphenicol medium per well. This plate will then be used for the pooling of the clones. These plates are incubated overnight 37° C.

Pooling the Clones

The “deep-well” plates are arranged in groups of 24.

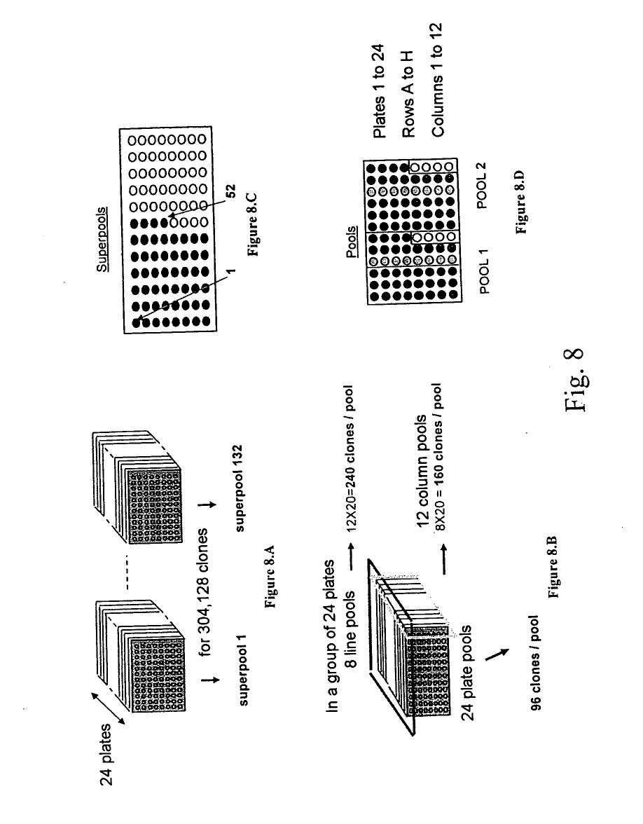

The first level of pooling is that of creating “superpools”. These correspond to the pooling of all the clones from a group of 24 plates. The number of superpools is given by the total number of plates in the library divided by 24. This system makes it possible to rapidly locate a clone in a group of 24 plates by carrying out a number of PCR stages corresponding to the number of superpools (FIG. 8.A).

The second level of pooling is that of creating pools. Still in groups of 24 plates, the clones are pooled by lines (line pools), by columns (column pools) and by plates (plate pools) as indicated in FIG. 8.B. If a clone is found in this group, one of the 24 plate pools should be positive in the PCR as well as a line pool and a column pool. Crosschecking using these three items of information gives the address of the clone in the library.

These pools are prepared using a Qiagen Biorobot3000 automated station or a Beckmann Biomek2000 automated station. These stations are pipetting robots which operate using sterile disposable tips.

Preparing the DNAs from the Pools

All the pools are centrifuged, after which the bacterial pellets are washed with Tris-EDTA (Sigma, ref. E5134) (TE), recentrifuged and then resuspended in TE. The DNA of the BACs is then recovered by boiling the pools in a microwave oven. The pools are then centrifuged and the supernatants are recovered.

Distributing the DNAs in 96-Well Plates for the PCR Screening

The supernatants from all the superpools are distributed in one or more 96-well deep-well plates in order to form a stock which will be easy to manipulate using a multichannel pipette (FIG. 8.C).

In the case of one pool, the 24 supernatants from the plate pools, the 12 supernatants from the column pools and the 8 supernatants from the line pools are distributed in half a 96-well deep-well plate (FIG. 8.D). It is possible to arrange two pools in each plate.

The DNA from each well can then be diluted for preparing plates which can be used for PCR.

### Example 5

### Constructing a Screening Vector and Performing Homologous Recombination on ES Cells

Ascertaining the Stability of the Mini-BACs in the Library

A representative sample of 40 clones was cultured and analyzed so as to ascertain the stability of the mini-BACs. No instability or decrease in the growth of the bacteria was observed as compared with the control pBe1oBac11 vector without insert.

Ascertaining the Size of the Inserts in the BAC Library

The plasmid DNAs from 40 clones were analyzed by enzyme digestion. The sizes of the inserts are all between 20 and 30 kb (as described in the optimum conditions for creating the rTgV library).

Constructing the Screening Vector

A mini-BAC containing a 20 kb insert was selected from these 40 clones. A selection gene was introduced by means of enzyme digestion and religation. The small homology arm (1.4 kb in the case of the positive control, 1.1 kb in the case of the RH vector) and the long homology arm (9 kb) were obtained by the action of exonuclease III (see protocol in Sambrook et al., chapter 5). A so-called positive control vector and a homologous recombination vector were thus constructed. A simple sequencing (of two times 500 bp) sufficed for working out the screening of the homologous recombination event.

Electroporation and Homologous Recombination in ES Cells

This step is performed using the methods known in the art. The level of homologous recombination in ES cells depends on the homologous sequences of the homologous recombination vector and not on the way it has been constructed.

### REFERENCES

Araki, et al. (1995). Proc Natl Acad Sci USA 92, 1604.

Austin, et al. (1981). Cell 25, 729-36.

Babinet, and Cohen-Tannoudji, (2000). médecine/science 16, 31-42.

Baron U. et al, (1999). PNAS 96: 1008-1013.

Brune, et al. (1999). Nat Biotechnol 17, 360-4.

Capecchi, (1989). Science 244, 1288-92.

Chatterjee, and Coren, (1997). Nucleic Acids Res 25, 2205-12.

Cohen-Tannoudji and Babinet, (1998). Mol Hum Reprod 4, 929-38.

Cohen-Tannoudji, et al. (1998). Mol Cell Biol 18, 1444-8.

Colleaux, et al. (1988). Proc Natl Acad Sci USA 85, 6022-6.

de Wit, et al. (1998). Nucleic Acids Res 26, 676-8.

Detloff, et al. (1994). Mol Cell Biol 14, 6936-43.

Evans, and Kaufman, (1981). Nature 292, 154-6.

Fiering, et al. (1999). Methods Enzymol 306, 42-66.

Goryshin, et al. (2000). Nat Biotechnol 18, 97-100.

Goryshin and Reznikoff, (1998). J Biol Chem 273, 7367-74.

Grant et al 1990. Proc. Natl. Acad. Sci. USA 87, 4645-4649.

Gu, et al. (1994). Science 265, 103-6.

Harada, et al. (1999). Embo J 18, 5931-42.

Hasty, and Bradley. (1993). Gene targeting vectors for mammalian. In Gene targeting: A pratical Approach, I. press, ed., pp. 1-31.

Hasty et al. (1991). Nature 350, 243-6.

Hasty, et al. (1991). Mol Cell Biol 11, 5586-91.

Hoess and Abremski (1985). J Mol Biol 181, 351-62.

Jaxson laboratory. The transgenic/Targeted Mutated Data base.

Kilby, et al. (1993). Trends Genet 9, 413-21.

Kulkarni, et al. (1999). Cell 96, 329-39.

laboratory, C. protocols/BAC library/sequences.

laboratory, C. protocols/pictures/pbe1oBAC.

laboratory, C. G. R. protocols/BAC library/construction.

Li, et al. (1997). J Cell Biol 139, 129-44.

Marshall, and Lemieux. (1991). Gene 104, 241-5.

Martin, (1981). Proc Natl Acad Sci USA 78, 7634-8.

Monteilhet, et al. (1990). Nucleic Acids Res 18, 1407-13.

Moore, et al. (1998). Nat Genet 18, 118-25.

Muyrers, et al. (1999). Nucleic Acids Res 27, 1555-7.

Sambrook, et al, 1989. Molecular Cloning: A Laboratory Manual, second ed. Cold Spring Harbor Laboratory Press, Cold Spring Harbor, N.Y.

Sauer, and Henderson, (1988). Proc Natl Acad Sci USA 85, 5166-70.

Schibler et al. (1998). Mamm Genome 9, 119-24.

Schlake, and Bode, (1994). Biochemistry 33, 12746-51.51.

Schneider-Maunoury, et al. (1993). Cell 75, 1199-214.

Sheng et al, 1995. Nucleic Acids Res. 23, 1990-1996.

Shibata, et al. (1997). Science 278, 120-3.

Smithies, (1993). Animal models of human genetic diseases. Trends Genet 9, 112-6.

Smithies, et al. (1985). Nature 317, 230-4.

Stellwagen, and Craig, (1997). Embo J 16, 6823-34.

Sternberg, et al. (1986). J Mol Biol 187, 197-212.

Sunaga, et al. (1997). Mol Reprod Dev 46, 109-13.

Tajbakhsh, et al. (1996). Nature 384, 266-70.

Thomas, and Capecchi, (1987). Cell 51, 503-12.

Thomas, et al. (1992). Mol Cell Biol 12, 2919-23.

Tsien et al. (1996). Cell 87, 1327-38.

Vaiman D; et al. (1999). Mammalian Genome 10, 585587

Westphal and Leder. (1997). Curr Biol 7, 530-3.

Wong, et al. (1986). Somat Cell Mol Genet 12, 63-72.

Woo, S. et al. (1994) Nucleic Acids Res. 22, 49224931

Xu et al. (1999). Nat Genet 22, 37-43.

Yagi, et al. (1990). Proc Natl Acad Sci USA 87, 9918-22.

Yang, et al. (1997). Nat Biotechnol 15, 859-65.

1. A cloning vector which is suitable for creating a genomic DNA library, characterized in that it exhibits a cassette comprising
a polylinker enabling a genomic DNA fragment to be integrated, with said polylinker being flanked by rare restriction sites, and in that said cassette is flanked by very rare restriction sites.

2. The vector as claimed in claim 1, characterized in that said cassette also comprises at least one negative selection gene.

3. The vector as claimed in claim 1 or 2, characterized in that said polylinker enabling a genomic DNA fragment to be integrated possesses a maximum of 4 restriction sites.

4. The vector as claimed in claim 1, characterized in that it is derived from a cosmid or from an artificial chromosome vector, preferably a bacterial artificial chromosome vector.

5. The vector as claimed in claim 4, characterized in that it is derived from the vector pBe1oBAC 11.

6. The vector as claimed in claim 4, characterized in that at least one restriction site selected from the sites which are present 1, 2 or 3 times in the skeleton of the parent vector is absent from this vector.

7. The vector as claimed in claim 5, characterized in that its skeleton no longer possesses at least one site selected from ApaI, BstEII, SacII, SfiI, SpeI, SphI, StuI, XhoI, BssHII, EcoRI, EcoRV, KpnI, NdeI, NotI, NruI, PvuI, SgrAI, XbaI, PstI, SalI and SmaI.

8. The vector as claimed in claim 7, characterized in that its skeleton no longer possesses a BstEII site.

9. The vector as claimed in claim 1, characterized in that said cassette possesses a very rare restriction site at one of its ends and two very rare restriction sites at the other end, with one of these two sites being identical to the site which is located at the other end of the cassette.

10. The vector as claimed in claim 1, characterized in that said polylinker is flanked by several (a number greater than 3) rare restriction sites selected, in particular, from PmeI, SgrAI, RsrII, ClaI, NotI, PacI, SrfI and NheI.

11. The cloning vector as claimed in claims 2, characterized in that said cassette comprises two copies of the same negative selection gene, with these copies flanking said polylinker and rare restriction sites.

12. The vector as claimed in claim 1, characterized in that it is the vector having the sequence SEQ ID No. 1.

13. A method for performing a targeted recombination in an organism starting with a genomic DNA library in a vector according to claims 1, comprising:
a) incubating a vector of said genomic library with at least one DNA fragment which comprises at least one transposon, with said transposon comprising a positive selection gene, where appropriate, a negative selection gene and/or a marker gene, with said selection genes being flanked, where appropriate, by the sites of action I of a site-specific recombinase I, and, optionally, at least one site of action II of a site-specific recombinase II which is different from the first recombinase I, with said site of action II not being located between said sites of action I of the first recombinase I, in the presence of an enzyme possessing a transposase activity for said transposon(s), such that said transposon(s) is/are transferred into said genomic fragment,
b) introducing said vector, which is, where appropriate, linearized and which is carrying said genomic fragment into which said transposon(s) is/are inserted, into a target host cell derived from said organism,
c) selecting homologous recombination events in said target cell by using the positive selection gene(s) carried by said transposon(s) and, where appropriate, the negative selection gene carried by said cloning vector.

14. The method as claimed in claim 13, characterized in that step c) is followed by a step d):
d) subjecting the cells which have been transformed by homologous recombination and selected in step c) to the action of the recombinase I in order to eliminate the positive and, where appropriate, negative selection gene(s) carried by the transposon(s).

15. The method as claimed in claim 13, characterized in that the target organism is a multicellular organism and in that said target cell derived from said organism is a stem cell.

16. The method as claimed in claim 15, characterized in that said organism belongs to the rodent genus, in particular to the murine species.

17. The method as claimed in claim 13, characterized in that the deletion of a genomic fragment is induced in said organism after step c) or, where appropriate, step d) by bringing the cells selected in step c), or derivatives of said cells, into contact with the recombinase II.

18. A kit comprising:
a cloning vector as claimed in claim 1,
at least one DNA fragment which comprises at least one transposon, with said transposon comprising a positive selection gene, where appropriate a negative selection gene and/or a marker gene, where appropriate flanked by the sites of action I of a site-specific recombinase I and optionally at least one site of action II for a site-specific recombinase II which is different from the first recombinase I, with said site of action II not being located between said sites of action I of the first recombinase I.

19. The kit as claimed in claim 18, characterized in that it comprises one or two DNA fragments, with each one comprising a transposon and with each transposon carrying a different positive selection gene and, where appropriate, a negative selection gene which is preferably identical in the case of the two transposons, with said positive and negative selection genes being flanked by the sites of action of a recombinase I.

20. The kit as claimed in claim 19, characterized in that each transposon comprises a sequence which corresponds to a site of action of a recombinase II which is different from the first recombinase I.

21. The kit as claimed in claims 18, characterized in that said recombinase I is the recombinase FLP, with said recombinase II being the recombinase Cre.

22. The kit as claimed in claim 18, characterized in that it comprises an enzyme which possesses a transposase activity for said transposon(s).

23. The kit as claimed in claim 18 for implementing a method as claimed in one of claims 13 to 17.

25. A genomic DNA library, characterized in that the genomic DNA fragments are cloned into a vector as claimed in claims 1.


SEQUENCE LISTING (4 sequences)

SEQ ID NO: 1 - DNA, 13478 bp, Artificial sequence
  Description of the artificial sequence  Vector rTgV, 13478 bp, circular
ctcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacgttacccaggccgtgccggcacgttaaccgggctgcatccgatgcaagtgtgtcgctgtcgagtaactataacggtcctaaggtagcgaggcgcgccatttgggctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaccatggaccctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctttgtgagaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcacaagctccctcgagggagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatctgtcgagggccggccgagctcatgcattgcggccgcgtttaaacatttaaatgtaatacgactcactatagggcgaggatccaagcttagtattctatagtgtcacctaaatcgtatgtcgaccggaccggcccgggcgcatgcttaattaatggcaaacagctattatgggtattatgggtctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcaccatggaccctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctttgtgagaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcacaagctccctcgagggagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatcgatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctggatcaaatctcgactcgctaccttaggaccgttatagtta

SEQ ID NO: 2 - DNA, 84 bp, Artificial sequence
  Description of the artificial sequence Sequence containing restriction sites
ctcgagtaactataacggtcctaaggtagcgaggcgcgccatcgatgtcgactcgctaccttaggaccgttatagttactcgag

SEQ ID NO: 3 - DNA, 22 bp, Artificial sequence
  Description of the artificial sequence Sequence containing restriction sites
ggcgcgccatttaaatctcgag

SEQ ID NO: 4 - DNA, 186 bp, Artificial sequence
  Description of the artificial sequence Sequence containing restriction sites
tcgagggccggccgagctcatgcattgcggccgcgtttaaacatttaaatgtaatacgactcactatagggcgaggatccaagcttagtattctatagtgtcacctaaatcgtatgtcgaccggaccggcccgggcgcatgcttaattaatggcaaacagctattatgggtattatgggtctcgag